(19) United States
(12) Patent Application Publication
(10) Pub. No.: US 2025/0229005 A1
(43) Pub. Date: Jul. 17, 2025

(54) OXIME-BASED CROSSLINKED HYDROGELS WITH ENHANCED RADIOPACITY FOR MEDICAL APPLICATIONS

(71) Applicant: Boston Scientific Scimed, Inc., Maple Grove, MN (US)

(72) Inventors: Yen-Hao Hsu, Shrewsbury, MA (US); Alex Kleinfehn, Plymouth, MN (US)

(73) Assignee: Boston Scientific Scimed, Inc., Maple Grove, MN (US)

(21) Appl. No.: 19/013,704

(22) Filed: Jan. 8, 2025

Related U.S. Application Data
(60) Provisional application No. 63/620,029, filed on Jan. 11, 2024.

(51) Int. Cl. A61L 27/52; A61L 27/44

(52) U.S. Cl. CPC A61L 27/52; A61L 27/44; A61L 2400/06

(57) ABSTRACT

In some aspects, the present disclosure provide systems for forming crosslinked reaction products, which comprise (a) a first composition that comprises a first multifunctional molecule that comprises a plurality of first reactive moieties that comprise oxyamine groups and (b) a second composition that comprises a second multifunctional molecule that comprises a plurality of second reactive moieties that comprise aldehyde or ketone groups, wherein at least one of the first and second multifunctional molecules is a multi-arm polymer and wherein at least one of the first and second multifunctional molecules comprises one or more covalently attached iodine groups. Other aspects of the present disclosure pertain to crosslinked networks that are formed using such systems and methods of treatment using such systems.

### CROSS-REFERENCE TO RELATED APPLICATIONS

The application claims the benefit of U.S. Provisional Patent Application Ser. No. 63/620,029 filed on Jan. 11, 2024, the disclosure of which is incorporated herein by reference.

### FIELD

The present disclosure relates to oxime-based radiopaque hydrogels and to crosslinkable systems for forming such radiopaque hydrogels, among other aspects. The hydrogels and crosslinkable systems for forming the same are useful, for example, in various medical applications.

### BACKGROUND

SpaceOAR®, a rapid crosslinking hydrogel that polymerizes in vivo within seconds, is based on a multi-arm polyethylene glycol (PEG) polymer functionalized with succinimidyl glutarate as reactive end groups which further react with trilysine to form crosslinks. This product has become a very successful, clinically-used biomaterial in prostate cancer therapy. A further improvement based on this structure is that a portion the succinimidyl glutarate end groups have been functionalized with 2,3,5-triiiodobenzamide groups, providing radiopacity. This hydrogel, known by the trade name of SpaceOAR Vue®, is the radiopaque version of SpaceOAR® for prostate medical applications. Above a specific pH, the succinimidyl glutarate groups will rapidly react with the trilysine crosslinker in vivo to form a hydrogel. The hydrogel breakdowns in vivo over the course of ca. 6-9 months. The breakdown occurs primarily through the hydrolysis of the ester linkages associated with the glutarate group.

### SUMMARY

The present disclosure provides an alternative approach to the SpaceOAR® products described above.

In some aspects, a system for forming a crosslinked reaction product is provided, which comprises (a) a first composition that comprises a first multifunctional molecule that comprises a plurality of first reactive moieties that comprise oxyamine groups and (b) a second composition that comprises a second multifunctional molecule that comprises a plurality of second reactive moieties that comprise aldehyde or ketone groups, wherein at least one of the first and second multifunctional molecules is a multi-arm polymer and wherein at least one of the first and second multifunctional molecules comprises one or more covalently attached iodine groups.

In some embodiments, the first reactive moieties further comprise hydrolysable linkages through which the oxyamine groups are linked to a remainder of the first multifunctional molecule and/or the second reactive moieties further comprise hydrolysable linkages through which the aldehyde or ketone groups are linked to a remainder of the first multifunctional molecule.

In some embodiments, which can be used in conjunction with the above aspects and embodiments, at least one of the first and second multifunctional molecules comprises covalently attached iodine groups in an iodinated core.

In some embodiments, which can be used in conjunction with the above aspects and embodiments, at least one of the first and second multifunctional molecules comprises covalently attached iodine groups in a residue of an iodo-substituted cyclic ester compound.

In some embodiments, which can be used in conjunction with the above aspects and embodiments, (a) the first multifunctional molecule is an iodinated multi-arm polymer having a core and a plurality of polymer arms that have oxyamine end groups and (b) the second multifunctional molecule is a non-iodinated multi-arm polymer having a core and a plurality of polymer arms that have aldehyde or ketone end groups, an iodinated multi-arm polymer having a core and a plurality of polymer arms that have aldehyde or ketone end groups, a non-polymeric molecule having a non-iodinated core and a plurality of pendant reactive moieties that comprise aldehyde or ketone groups, or a non-polymeric molecule having an iodinated core and a plurality of pendant reactive moieties that comprise aldehyde or ketone groups.

In some embodiments, which can be used in conjunction with the above aspects and embodiments, (a) the first multifunctional molecule is a non-iodinated multi-arm polymer having a core and a plurality of polymer arms that have oxyamine end groups and (b) the second multifunctional molecule is an iodinated multi-arm polymer having a core and a plurality of polymer arms that have aldehyde or ketone end groups or a non-polymeric molecule having an iodinated core and a plurality of pendant reactive moieties that comprise aldehyde or ketone groups.

In some embodiments, which can be used in conjunction with the above aspects and embodiments, (a) the first multifunctional molecule is a non-polymeric molecule having an iodinated core and a plurality of pendant reactive moieties that comprise oxyamine groups and (b) the second multifunctional molecule is a non-iodinated multi-arm polymer having a core and a plurality of polymer arms that have aldehyde or ketone end groups or an iodinated multi-arm polymer having a core and a plurality of polymer arms that have aldehyde or ketone end groups.

In some embodiments, which can be used in conjunction with the above aspects and embodiments, (a) the first multifunctional molecule is a non-polymeric molecule having a non-iodinated core and a plurality of pendant reactive moieties that comprise oxyamine groups and (b) the second multifunctional molecule is an iodinated multi-arm polymer having a core and a plurality of polymer arms that have aldehyde or ketone end groups.

In some embodiments, which can be used in conjunction with the above aspects and embodiments, the iodinated core comprises one or more iodinated aromatic groups.

In some embodiments, which can be used in conjunction with the above aspects and embodiments, the iodinated core comprises an iodinated polyol residue.

In some embodiments, which can be used in conjunction with the above aspects and embodiments, the system comprises the first composition in a first container and the second composition in a second container. In some of these embodiments, the first and second containers are independently selected from vials and syringes and/or the system further comprises a delivery device that is configured to deliver the first and second compositions. The device may be, for example, a delivery device that comprises a dual barrel syringe.

In other aspects, the present disclosure pertains to a crosslinked network formed by combining (a) a first multifunctional molecule that comprises a plurality of first reactive moieties that comprise oxyamine groups and (b) a second multifunctional molecule that comprises a plurality of second reactive moieties that comprise aldehyde or ketone groups, wherein at least one of the first and second multifunctional molecules is a multi-arm polymer and wherein at least one of the first and second multifunctional molecules comprises one or more covalently attached iodine groups.

In some embodiments, the crosslinked network is a hydrogel.

In some embodiments, which can be used in conjunction with the above aspects and embodiments the crosslinked network has a radiopacity that is greater than 250 Hounsfield units.

In some embodiments, which can be used in conjunction with the above aspects and embodiments, the crosslinked network is in the form of a suspension of particles of the crosslinked network. In some of these embodiments, the suspension of particles is provided in a syringe barrel.

In other aspects, the present disclosure pertains to a method of treatment comprising administering to a subject a mixture that comprises (a) a first multifunctional molecule that comprises a plurality of first reactive moieties that comprise oxyamine groups and (b) a second multifunctional molecule that comprises a plurality of second reactive moieties that comprise aldehyde or ketone groups, wherein at least one of the first and second multifunctional molecules is a multi-arm polymer and wherein at least one of the first and second multifunctional molecules comprises one or more covalently attached iodine groups.

Potential benefits of the present disclosure include the following: enhanced in vivo persistence and/or X-ray visibility, good homogeneity, and rapid and efficient hydrogel formation.

The above and other aspects, embodiments, features and benefits of the present disclosure will be readily apparent from the following detailed description.

### BRIEF DESCRIPTION OF THE DRAWINGS

FIG. 1 schematically illustrates a method of forming hydroxyl-terminated multi-arm PEG, in accordance with an embodiment of the present disclosure.

FIG. 2 schematically illustrates a method of forming iodinated hydroxyl-terminated multi-arm PEG, in accordance with an embodiment of the present disclosure.

FIG. 3 schematically illustrates a method of forming an iodinated ketone-terminated multi-arm PEG from the hydroxyl-terminated multi-arm PEG of FIG. 1A, in accordance with an embodiment of the present disclosure.

FIG. 4 schematically illustrates a method of forming an iodinated oxyamine-terminated multi-arm PEG from the iodinated hydroxyl-terminated multi-arm PEG of FIG. 2, in accordance with an embodiment of the present disclosure.

FIG. 5 schematically illustrates a method of forming an iodinated multifunctional oxyamine compound, in accordance with an embodiment of the present disclosure.

FIG. 6 schematically illustrates the formation of a crosslinked product resulting from a crosslinking reaction between the iodinated ketone-terminated multi-arm PEG of FIG. 3 and the iodinated oxyamine-terminated multi-arm PEG of FIG. 4, in accordance with an embodiment of the present disclosure.

FIG. 7 schematically illustrates an iodinated oxyamine-terminated multi-arm PEG, in accordance with an embodiment of the present disclosure.

FIG. 8 schematically illustrates an iodinated ketone-terminated multi-arm PEG, in accordance with an embodiment of the present disclosure.

FIG. 9 schematically illustrates a delivery device, in accordance with an embodiment of the present disclosure.

FIG. 10 schematically illustrates a delivery device, in accordance with another embodiment of the present disclosure.

### DETAILED DESCRIPTION

In some aspects, the present disclosure provides systems that comprise (a) a first multifunctional molecule that comprises a plurality of first reactive moieties that comprise oxyamine groups and (b) a second multifunctional molecule that comprises a plurality of second reactive moieties that comprise aldehyde or ketone groups, wherein at least one of the multifunctional molecules is a multi-arm polymer and wherein at least one of the multifunctional molecules comprises one or more covalently attached iodine groups, for example, in an iodinated core or at an end of polymer arms of a multi-arm polymer.

The oxyamine groups of the first multifunctional molecule and the aldehyde or ketone groups of the second multifunctional molecule can react via a click reaction to form oxime bonds. In this way, such systems can be used to form radiopaque crosslinked reaction products, including radiopaque hydrogels, held together by oxime-containing linkages.

As used herein, a “hydrogel” is a crosslinked polymer that contains water or can absorb water but does not dissolve when placed in water.

In some embodiments, the first reactive moieties that comprise oxyamine groups further comprise a hydrolysable linkage, such as a hydrolysable ester linkage or a carbonate linkage, through which the oxyamine groups are linked to a remainder of the first multifunctional molecule. Examples of such first reactive moieties include first reactive moieties that comprise aminooxy-C₁-C₄-alkyl-carbonyloxy groups and aminooxy-C₁-C₄-alkyl-oxycarbonyl groups, among others.

In some embodiments, the second reactive moieties that comprise aldehyde or ketone groups, further comprise a hydrolysable linkage, such as a hydrolysable ester linkage or a carbonate linkage, through which the aldehyde or ketone groups are linked to a remainder of the second multifunctional molecule. Examples of such second reactive moieties include aldehyde-containing reactive moieties such as C₁-C₄-alkanal-carbonyloxy groups and C₁-C₄-alkanal-oxycarbonyl groups and ketone-containing reactive moieties such as C₂-C₈-alkenone-carbonyloxy groups and C₂-C₈-alkenone-oxycarbonyl groups.

In some embodiments, the first reactive moieties and/or the second reactive moieties further comprise one or more iodine groups, for example, as described further below.

Multi-arm polymers for use in conjunction with the present disclosure include multi-arm polymers that comprise (a) a core regain, (b) multiple polymer arms, each comprising at least one polymer segment linked to the core region and (c) at least one terminal reactive moiety linked to each of the polymer arms, the terminal reactive moiety selected from either a first reactive moiety that comprise an oxyamine group or a second reactive moiety that comprises an aldehyde or ketone group. In various embodiments, a first end of the polymer segment is linked to the core region and a second end of the polymer segment opposite the first end of the polymer segment is linked to the terminal reactive moiety. In some embodiments, the core region comprises a polyol residue. In some embodiments, the core region comprises an iodinated polyol residue.

Polymer segments for the polymer arms may be selected from any of a variety of synthetic, natural, or hybrid synthetic-natural polymer segments. Examples of polymer segments include those that are formed from one or more monomers selected from the following: C₁-C₆-alkylene oxide monomers (e.g., ethylene oxide, propylene oxide, tetramethylene oxide, etc.), cyclic ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), and 2-phenyl-2-oxazoline, polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, N-isopropylacrylamide, amino acids and sugars.

Polymer segments for the polymer arms may be selected, for example, from the following polymer segments: polyether segments including poly(C₁-C₆-alkylene oxide) segments such as poly(ethylene oxide) (PEO) segments (also referred to as polyethylene glycol segments or PEG segments), poly(propylene oxide) (PPO) segments, poly(ethylene oxide-co-propylene oxide) segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(γ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, poly(N-isopropylacrylamide) segments, polypeptide segments, and polysaccharide segments.

Each polymer segment for use in the multi-arm polymers of the present disclosure may contain between 2 and 1000 monomer units or more, for example, ranging anywhere from 2 to 5 to 10 to 30 to 70 to 100 to 300 to 700 to 1000 units (in other words, ranging between any two of the preceding values).

Polyol residues in accordance with the present disclosure include residues of polyols having three more hydroxyl groups, for example, containing anywhere from 3 to 100 hydroxyl groups (e.g., having 3 to 4 to 5 to 6 to 7 to 8 to 9 to 10 to 12 to 15 to 20 to 25 to 30 to 40 to 50 to 60 to 70 to 80 to 90 to 100 hydroxyl groups). General classes of polyols include sugars (monosaccharides, disaccharides, trisaccharides, etc.), sugar alcohols, calixaranes, polyhedral oligomeric silsesquioxanes (POSS), cyclodextrins, polyhydroxylated polymers, catechins, flavanols, anthocyanins, stilbenes, and polyphenols, among others.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, polyhedral oligomeric silsesquioxanes (POSS), catechins, flavanols, anthocyanins, stilbenes, polyphenols, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

In some embodiments, iodinated polyols may be employed to provide the resulting multifunctional molecule with radiopacity. Iodinated polyols include iodinated aromatic polyols, examples of which are compounds that comprise 3 or more hydroxyl groups, and one or more iodinated aromatic groups. Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with the three or more hydroxyl groups, which may be directly substituted to the aromatic groups or may be provided in the form of hydroxyalkyl groups (e.g., C₁-C₄-hydroxyalkyl groups containing one, two, three or four carbon atoms and containing one, two, three or four or more hydroxyl groups). The hydroxyalkyl groups may be linked to the aromatic group directly or through any suitable linking moiety, which may be selected, for example, from amide groups, amine groups, ether groups, ester groups, or carbonate groups, among others.

Specific examples of iodinated polyols for use in the present disclosure include iodinated polyols that are known for use as iodinated contrast agents, whose biocompatibility has been demonstrated to be reasonably well tolerated. Specific examples of iodinated polyols include commercially available 1,3,5-triiodo-2,4,6-trishydroxymethylbenzene (CAS #178814-33-0),

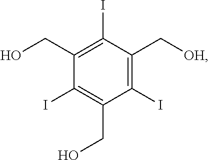 [SMILES: OCc1c(I)c(CO)c(I)c(CO)c1I]

iodixanol (CAS #92339-11-2),

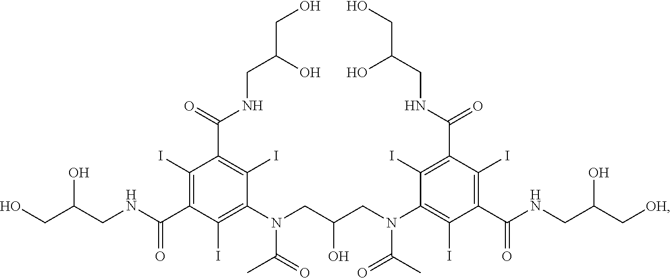 [SMILES: CC(=O)N(CC(O)CN(C(C)=O)c1c(I)c(C(=O)NCC(O)CO)c(I)c(C(=O)NCC(O)CO)c1I)c1c(I)c(C(=O)NCC(O)CO)c(I)c(C(=O)NCC(O)CO)c1I]

iotrolan (CAS #79770-24-4),

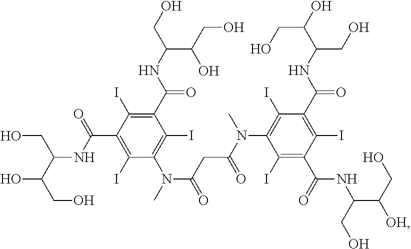 [SMILES: CN(C(=O)CC(=O)N(C)c1c(I)c(C(=O)NC(CO)C(O)CO)c(I)c(C(=O)NC(CO)C(O)CO)c1I)c1c(I)c(C(=O)NC(CO)C(O)CO)c(I)c(C(=O)NC(CO)C(O)CO)c1I]

iohexol (CAS #66108-95-0),

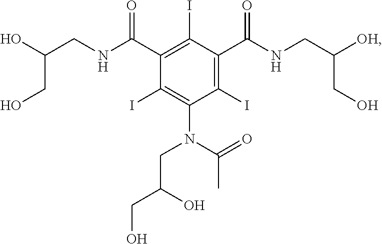 [SMILES: CC(=O)N(CC(O)CO)c1c(I)c(C(=O)NCC(O)CO)c(I)c(C(=O)NCC(O)CO)c1I]

ioversol (CAS #87771-40-2),

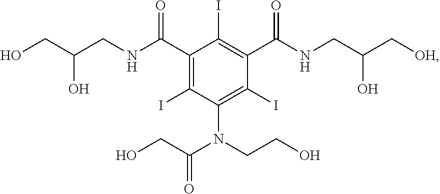 [SMILES: O=C(NCC(O)CO)c1c(I)c(C(=O)NCC(O)CO)c(I)c(N(CCO)C(=O)CO)c1I]

iopamidol (CAS #60166-93-0),

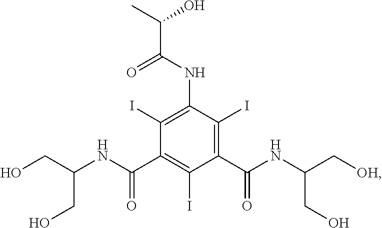 [SMILES: C[C@H](O)C(=O)Nc1c(I)c(C(=O)NC(CO)CO)c(I)c(C(=O)NC(CO)CO)c1I]

iohexol impurity J (CAS #76801-93-9),

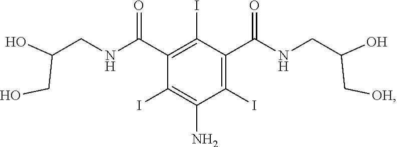 [SMILES: Nc1c(I)c(C(=O)NCC(O)CO)c(I)c(C(=O)NCC(O)CO)c1I]

and iopromide (CAS #73334-07-3),

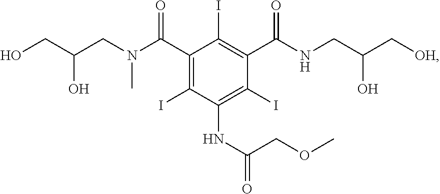 [SMILES: COCC(=O)Nc1c(I)c(C(=O)NCC(O)CO)c(I)c(C(=O)N(C)CC(O)CO)c1I]

among others.

In some embodiments of the present disclosure, preformed polymer segments may be attached to the above polyols. For example, carboxyl terminated polymer segments may be attached to hydroxyl groups of the polyols through an ester coupling reaction.

In some embodiments of the present disclosure, non-iodinated or iodinated polyols such as those described above are used as multi-functional initiators for polymer chain growth, leading to hydroxy terminated multi-arm polymers. For example, the non-iodinated or iodinated polyols may be used as initiators for ring-opening polymerization of ethylene oxide to form poly(ethylene oxide) (PEG) segments at each of the hydroxyl groups of the polyol. The resulting hydroxy-terminated PEG segments possess tunable hydrophilicity depending on the desired water-solubility of the resulting multi-arm polymer, for example, with increasing PEG segment length leading to increasing hydrophilicity.

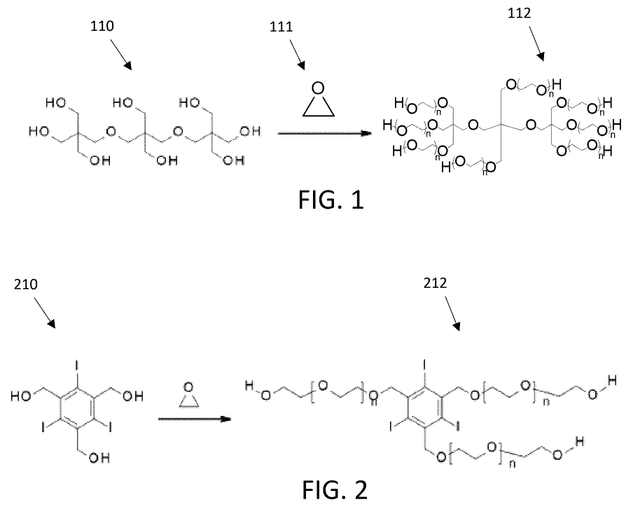

In a particular embodiment shown in FIG. 1, commercially available tripentaerythritol (110) (CAS #78-24-0) can be used as an octa-functional initiator, which undergoes ring-opening polymerization with ethylene oxide (111). The polymerization process leads to poly(ethylene oxide) (PEG) chain growth at each of the eight hydroxyl groups of the tripentaerythritol, forming a hydroxy-terminated 8-arm-PEG (112) having a tripentaerythritol residue core. In FIG. 1, n is an integer representing the number of monomer units in each polymer segment shown. In other cases, only a single monomer unit is present and n=1.

The strategy shown in FIG. 1 is broadly applicable and can be used in conjunction with a range of polyols, including those described above. In a particular embodiment shown in FIG. 2, an iodinated polyol, 1,3,5-triiodo-2,4,6-tris-hydroxymethylbenzene (210), is used as an initiator, which undergoes ring-opening polymerization with ethylene oxide. The polymerization process leads to poly(ethylene oxide) (PEG) chain growth at each of the three hydroxyl groups of the 1,3,5-triiodo-2,4,6-tris-hydroxymethylbenzene (210). The resulting multi-arm polymer (212) contains three PEG arms that extend from a 1,3,5-triiodo-2,4,6-tris-hydroxymethylbenzene residue core. Each of the PEG arms has a terminal hydroxyl group. In FIG. 2, n is an integer representing the number of monomer units in each polymer segment shown. In other cases, only a single monomer unit is present and n=1.

In some embodiments, where an ester is desired as a hydrolysable linkage, terminal hydroxyl groups of hydroxy-terminated multi-arm polymers such as those produced in conjunction with FIG. 1 or FIG. 2, among many others, may be reacted with a lactone (e.g., α-acetolactone, β-propiolactone, γ-butyrolactone, and δ-valerolactone, ε-caprolactone, etc.) to form a hydroxyl-terminated multi-arm polymer comprising a hydroxyl end group that is linked to a polymer segment through a hydrolysable ester group.

In other embodiments where an ester is desired as a hydrolysable linkage, terminal hydroxyl groups of hydroxy-terminated multi-arm polymers such as those produced in conjunction with FIG. 1 or FIG. 2, among many others, may be reacted with a cyclic anhydride (e.g., glutaric anhydride, succinic anhydride, malonic anhydride, adipic anhydride, diglycolic anhydride, 1,3-acetonedicarboxylic acid anhydride, etc.) to form an acid-end-capped polymer segment such as a glutaric-acid-end-capped segment, a succinic-acid-end-capped segment, a malonic-acid-end-capped segment, an adipic-acid-end-capped segment, a diglycolic-acid-end-capped segment, a 1,3-acetonedicarboxylic-acid-end-capped segment, and so forth. The preceding cyclic anhydrides, among others, may be reacted with a hydroxy-terminated multi-arm polymer under basic conditions to form a carboxylic-acid-terminated multi-arm polymer comprising a carboxylic acid end group that is linked to a polymer segment through a hydrolysable ester group. Subsequently, the carboxylic acid end groups may be reduced to hydroxyl groups using a suitable reducing agent such as borane (BH₃).

In some embodiments, one or more iodine groups can be introduced at the same time as the hydrolysable ester group. For example, terminal hydroxyl groups of hydroxy-terminated multi-arm polymers such as those produced in conjunction with FIG. 1 or FIG. 2, among many others, may be reacted with an iodo-substituted cyclic ester compound such as an iodo-substituted dihydrobenzofuranone compound or an iodo-substituted dihydrobenzopyranone compound. Examples of iodo-substituted dihydrobenzofuranone compounds include monoiodo-dihydrobenzofuranones, diiodo-dihydrobenzofuranones, triiodo-dihydrobenzofuranones, and tetraiodo-dihydrobenzofuranones such as monoiodo-2,3-dihydro-1-benzofuran-2-ones, diiodo-2,3-dihydro-1-benzofuran-2-ones, triiodo-2,3-dihydro-1-benzofuran-2-ones, tetraiodo-2,3-dihydro-1-benzofuran-2-ones, monoiodo-1,3-dihydro-2-benzofuran-1-ones, diiodo-1,3-dihydro-2-benzofuran-1-ones, triiodo-1,3-dihydro-2-benzofuran-1-ones, and tetraiodo-1,3-dihydro-2-benzofuran-1-ones. Examples of iodo-substituted dihydrobenzopyranone compounds include iodo-substituted 3,4-dihydrobenzopyranones, such as monoiodo-3,4-dihydro-2H-1-benzopyran-2-ones, diiodo-3,4-dihydro-2H-1-benzopyran-2-ones, triiodo-3,4-dihydro-2H-1-benzopyran-2-ones, and tetraiodo-3,4-dihydro-2H-1-benzopyran-2-ones, monoiodo-3,4-dihydro-1H-2-benzopyran-1-ones, diiodo-3,4-dihydro-1H-2-benzopyran-1-ones, triiodo-3,4-dihydro-1H-2-benzopyran-1-ones, and tetraiodo-3,4-dihydro-1H-2-benzopyran-1-ones. Specific examples include 5-iodo-2(3H)-benzofuranone, also referred to as 5-iodo-2,3-dihydro-1-benzofuran-2-one,

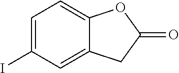 [SMILES: O=C1Cc2cc(I)ccc2O1]

(CAS #742079-78-3) and 7-iodo-3,4-dihydro-1H-2-benzopyran-1-one

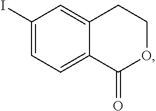 [SMILES: O=C1OCCc2cc(I)ccc21]

5,7-Diiodo-2(3H)-benzofuranone,

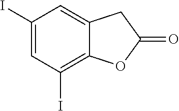 [SMILES: O=C1Cc2cc(I)cc(I)c2O1]

(CAS #2101526-57-0), 6-Iodo-2(3H)-benzofuranone,

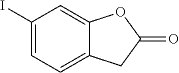 [SMILES: O=C1Cc2ccc(I)cc2O1]

(CAS #742079-91-0), 3,4-Dihydro-5-iodo-1H-2-benzopyran-1-one,

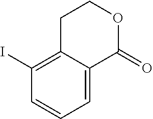 [SMILES: O=C1OCCc2c(I)cccc21]

(CAS #1823892-40-5), 3,4-Dihydro-7-iodo-1H-2-benzopyran-1-one,

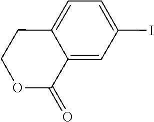 [SMILES: O=C1OCCc2ccc(I)cc21]

(CAS #1823368-75-7), and 3,4-Dihydro-8-iodo-1H-2-benzopyran-1-one,

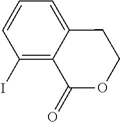 [SMILES: O=C1OCCc2cccc(I)c21]

### (CAS #1823368-80-4).

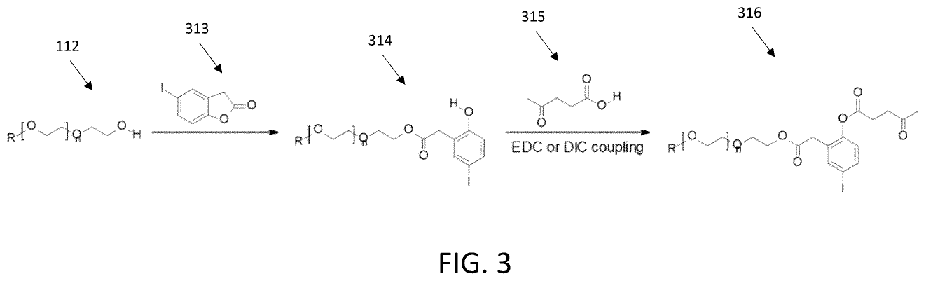

In a particular embodiment shown in FIG. 3, a hydroxyl terminated multi-arm polymer, specifically, the hydroxy-terminated 8-arm-PEG (112) of FIG. 2, where R represents a remainder of the molecule (including the tripentaerythritol residue core and the remaining 7 arms of the molecule) and n is an integer representing the number of monomer units in the polymer segment (or n may be 1 in some cases), may undergo a ring-opening reaction with an iodo-substituted cyclic ester compound, specifically, 5-iodo-2,3-dihydro-1-benzofuran-2-one (313), to produce a multi-arm polymer (314) in which each arm comprises a terminal moiety comprising a hydroxy group and an iodine group that is linked to a reminder of the arm by a hydrolysable ester group.

Hydroxy-terminated multi-arm polymers such as those described above among many others, may be used to form multi-arm polymers that comprise a plurality of reactive moieties that comprise aldehyde or ketone groups in accordance with the present disclosure and, in some embodiments, multi-arm polymers that comprise reactive moieties that comprise an aldehyde or ketone groups and a hydrolysable ester group through which the aldehyde or ketone groups are linked to a remainder of the multi-arm polymer.

For example, a compound that comprises an aldehyde or ketone group and a carboxylic acid group may be reacted with a hydroxy-terminated multi-arm polymer in an ester coupling reaction to form an aldehyde- or ketone-terminated multi-arm polymer. The ester coupling between the carboxylic acid and hydroxyl groups may be performed in the presence of a suitable coupling agent, for example, a carbodiimide coupling agent such as 1-ethyl-3-(3-dimethylaminopropyl)carbodiimide (EDC), N,N′-diisopropylcarbodiimide (DIC) or N,N′-dicyclohexylcarbodiimide (DCC). The ester linkage that is formed is hydrolysable.

Examples of compounds that comprise an aldehyde or ketone group and a carboxylic acid group include various C₃-C₈-acid aldehydes and C₃-C₈-acid ketones, including C₃-C₈-oxoalkanoic acids, such as 3-oxopropanoic acid, 3-oxobutanoic acid, 4-oxobutanoic acid, 3-oxopentanoic acid, 4-oxopentanoic acid, 5-oxopentanoic acid, 3-oxohexanoic acid, 4-oxohexanoic acid, 5-oxohexanoic acid, and 6-oxohexanoic acid.

In a particular example shown in FIG. 3, the iodinated hydroxy-terminated 8-arm-PEG (314) may be coupled with 4-oxopentanoic acid (CAS #123-76-2) (315) in the presence of an EDC or DIC coupling agent to obtain a ketone-terminated 8-Arm-PEG polymer (316), where R represents a remainder of the molecule (including the tripentaerythritol residue core and the remaining 7 arms of the molecule) and n is an integer representing the number of monomer units in each polymer segment. In other cases, n=1.

The strategy shown in FIG. 3 is widely applicable to other hydroxy-terminated polymers. For example, in another embodiment, the iodinated hydroxy-terminated 3-arm PEG of FIG. 2 may likewise undergo a ring-opening reaction with an iodo-substituted cyclic ester compound, specifically, 5-iodo-2,3-dihydro-1-benzofuran-2-one (313), followed by ester coupling with 4-oxopentanoic acid (315) to obtain an iodinated ketone-terminated 3-arm-PEG polymer (816) as shown in FIG. 8 where n is an integer representing the number of monomer units in each polymer segment. In other cases, n=1.

In other embodiments where an iodinated product is not needed, the ring-opening reaction with an iodo-substituted cyclic ester compound may be eliminated and a hydroxy-terminated multi-arm polymer such as that of FIG. 1, among many others, may be directly reacted with a compound that comprises an aldehyde or ketone group and a carboxylic acid group to form an aldehyde- or ketone-terminated multi-arm polymer.

In other embodiments, multifunctional compounds having aldehyde or ketone groups in accordance with the present disclosure may be formed directedly from polyols, including non-iodinated or iodinated polyols such as those described above, without the formation of an intervening polymer arm. These resulting multifunctional molecules are non-polymeric in nature and comprise a non-iodinated or iodinated polyol residue core with multiple pendant aldehyde or ketone groups.

For example, the carboxylic acid group of a compound comprising an aldehyde or ketone group and a carboxylic acid group may be reacted with the hydroxyl groups of a non-iodinated or iodinated polyol in an ester coupling reaction to form a multifunctional molecule that comprises a polyol residue with multiple aldehyde or ketone pendant groups formed at the locations of the reacted hydroxyl groups. As above, the ester coupling between the carboxylic acid and the hydroxyl groups may be performed in the presence of a suitable coupling agent, for example, a carbodiimide coupling agent such as EDC, DCC or DIC. The ester linkage that is formed may be subject to hydrolysis.

Hydroxy-terminated multi-arm polymers such as those described above may also be used to form multi-arm polymers that comprises a plurality of reactive moieties that comprise oxyamine groups in accordance with the present disclosure, for example, multifunctional molecules that comprises a polyol residue core and a plurality PEG arms each terminated with an oxyamine group, in accordance with the present disclosure.

In some embodiments, hydroxyl groups of a hydroxy-terminated multi-arm polymer may first be reacted in an ester coupling reaction with a carboxyl group of a protected compound that comprises an oxyamine group and a carboxyl group. The ester coupling between the carboxylic acid and the hydroxyl groups may be performed in the presence of a suitable coupling agent, for example, a carbodiimide coupling agent such as EDC, DCC or DIC. The ester linkage that is formed may be subject to hydrolysis. After ester coupling, the resulting protected-oxyamine-terminated multi-arm polymer is then deprotected to remove the protective groups and form an oxamine-terminated multi-arm polymer.

Examples of compounds that comprise an oxyamine group and a carboxyl group include various C₂-C₈-(aminooxy)alkanoic acids, such as aminooxyacetic acid, 2-(aminooxy)propanoic acid, 3-(aminooxy)propanoic acid, 2-(aminooxy)butanoic acid, 3-(aminooxy)butanoic acid, 4-(aminooxy)butanoic acid, 2-(aminooxy)pentanoic acid, 3-(aminooxy)pentanoic acid, 4-(aminooxy)pentanoic acid, 5-(aminooxy)pentanoic acid, 2-(aminooxy)hexanoic acid, 3-(aminooxy)hexanoic acid, 4-(aminooxy)hexanoic acid, 5-(aminooxy)hexanoic acid, and 6-(aminooxy)hexanoic acid.

Prospective protective chemistries for the oxyamine group include tert-butoxycarbonyl (Boc) groups, carboxybenzyl (Cbz) groups, 6-nitroveratryloxycarbonyl (Nvoc) groups, 9-fluorenylmethoxycarbonyl (Fmoc) groups, triphenylmethylamine (Tritylamine) (Tr-NR2) groups, and benzylideneamine groups. Protective groups may be removed, for example, by exposure to an acid such as HCl or trifluoroacetic acid.

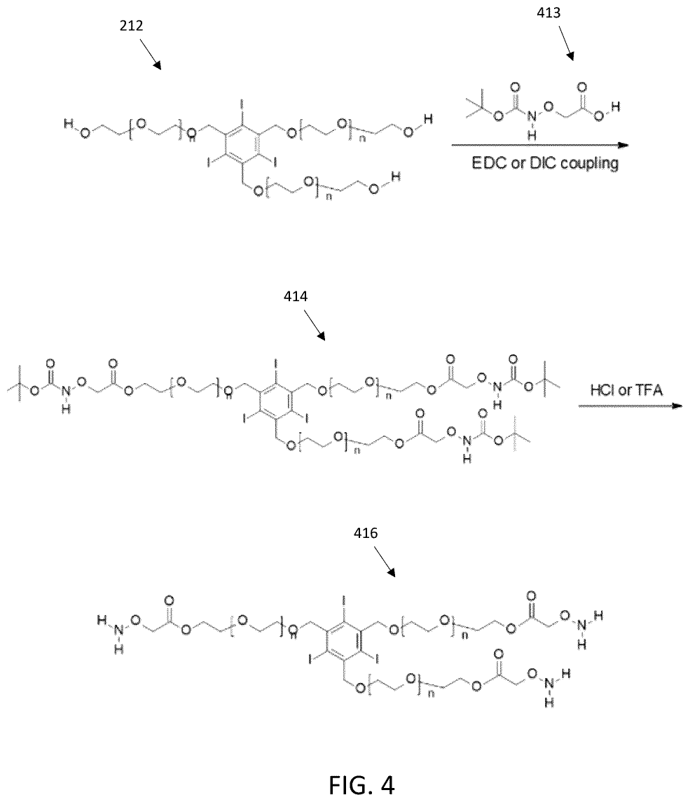

In a particular example shown in FIG. 4, the iodinated hydroxy-terminated 3-arm PEG (212) of FIG. 2 is reacted with (Boc-aminooxy)acetic acid (413), also known as N-Boc-(carboxymethoxy)amine (CAS #42989-85-5) in the presence of EDC or DIC as a coupling agent to obtain an iodinated Boc-aminooxy-methylcarbonyloxy-terminated 3-arm PEG (414), which is then deprotected using HCl or trifluoroacetic acid (TFA), thereby forming an iodinated oxyamine-terminated 3-arm PEG (416).

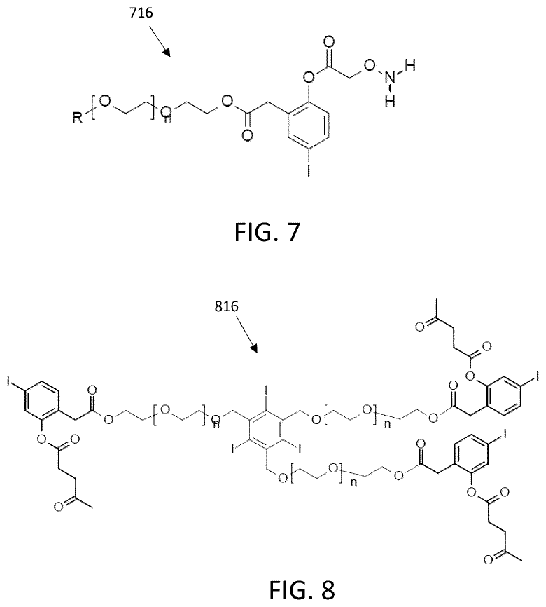

The strategy shown in FIG. 4 is widely applicable to other hydroxy-terminated polymers. For example, in another embodiment, the iodinated hydroxy-terminated 8-arm PEG (313) of FIG. 3 may likewise be reacted with (Boc-aminooxy)acetic acid, which is subsequently deprotected to form an iodinated oxyamine-ester-terminated 8-arm-PEG polymer (716) as shown in FIG. 7, where R represents a remainder of the molecule (including the tripentaerythritol residue core and the remaining 7 arms of the molecule) and n is an integer representing the number of monomer units in each polymer segment. In other cases, n=1.

In other embodiments, multifunctional molecules that comprise oxyamine groups in accordance with the present disclosure are formed directedly from polyols, including non-iodinated or iodinated polyols such as those described above, without the formation of n intervening polymer arm. These resulting multifunctional molecules are thus generally non-polymeric in nature and comprise a non-iodinated or iodinated polyol residue core with multiple pendant oxyamine groups.

For example, hydroxyl groups of the polyol may first be reacted in an ester coupling reaction with a carboxyl group of a protected compound that comprises an oxyamine group and a carboxyl group. As above, the ester coupling between the carboxylic acid and the hydroxyl groups may be performed in the presence of a suitable coupling agent, for example, a carbodiimide coupling agent such as EDC, DCC or DIC. The ester linkage that is formed may be subject to hydrolysis. After ester coupling, the resulting multifunctional molecule, which comprises a polyol residue with pendant moieties containing protected oxyamine groups, is then deprotected remove the protective groups and form a multifunctional molecule that comprises a polyol residue with pendant reactive moieties containing oxyamine ester groups formed at the locations of the previously reacted hydroxyl groups.

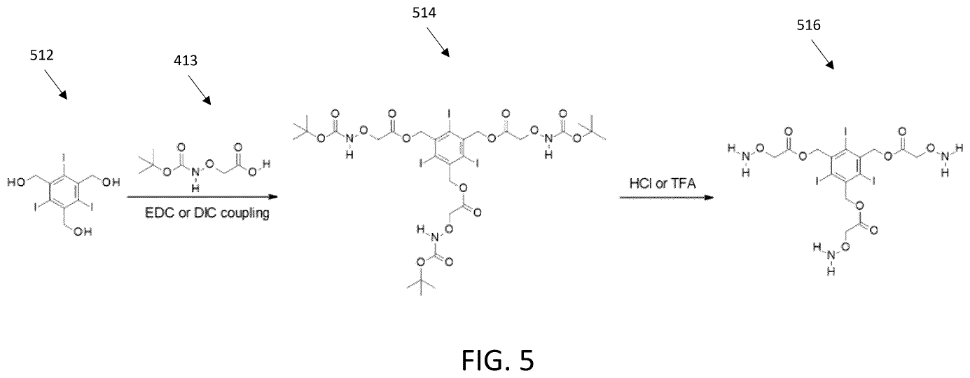

In a particular example shown in FIG. 5, commercially available 1,3,5-triiodo-2,4,6-trishydroxymethylbenzene (512) is reacted with is reacted with (Boc-aminooxy)acetic acid (413), forming an intermediate compound (514) that comprises a 1,3,5-triiodo-2,4,6-trishydroxymethylbenzene residue with pendant moieties containing protected oxyamine ester groups formed at the locations of the reacted hydroxyl groups. The intermediate compound (514) is then deprotected with HCl or TFA to form a multifunctional iodinated oxyamine compound that comprises a 1,3,5-triiodo-2,4,6-trishydroxymethylbenzene residue with pendant oxyamine ester groups formed at the locations of the reacted hydroxyl groups.

The above and other techniques may be used to form the following: a first multifunctional molecule in the form of a non-iodinated multi-arm polymer having a core and a plurality of polymer arms that have oxyamine end groups, a first multifunctional molecule in the form of an iodinated multi-arm polymer having a core and a plurality of polymer arms that have oxyamine end groups, a first multifunctional molecule in the form of a non-polymeric molecule having a non-iodinated core and a plurality of pendant reactive moieties that comprise oxyamine groups, a first multifunctional molecule in the form of a non-polymeric molecule having an iodinated core and a plurality of pendant reactive moieties that comprise oxyamine groups, a second multifunctional molecule in the form of a non-iodinated multi-arm polymer having a core and a plurality of polymer arms that have aldehyde or ketone end groups, a second multifunctional molecule in the form of an iodinated multi-arm polymer having a core and a plurality of polymer arms that have aldehyde or ketone end groups, a second multifunctional molecule in the form of a non-polymeric molecule having a non-iodinated core and a plurality of pendant reactive moieties that comprise aldehyde or ketone groups, and a second multifunctional molecule in the form of a non-polymeric molecule having an iodinated core and a plurality of pendant reactive moieties that comprise aldehyde or ketone groups.

As noted above, in some aspects, the present disclosure provides systems that comprise (a) a first multifunctional molecule that comprises a plurality of reactive moieties that comprise oxyamine groups and (b) a second multifunctional molecule that comprises a plurality of reactive moieties that comprise aldehyde or ketone groups, wherein at least one of the first and second multifunctional molecules is a reactive multi-arm polymer and wherein at least one of the first and second multifunctional molecules comprises one or more covalently attached iodine groups.

For example, the system may comprise (a) first multifunctional molecule in the form of a non-iodinated multi-arm polymer having a core and a plurality of polymer arms that have oxyamine end groups and (b) a second multifunctional molecule in the form of a non-polymeric molecule having an iodinated core and a plurality of pendant reactive moieties that comprise aldehyde or ketone groups.

As another example, the system may comprise (a) first multifunctional molecule in the form of a non-iodinated multi-arm polymer having a core and a plurality of polymer arms that have oxyamine end groups and (b) a second multifunctional molecule in the form of an iodinated multi-arm polymer having a core and a plurality of polymer arms that have aldehyde or ketone end groups.

As another example, the system may comprise (a) first multifunctional molecule in the form of an iodinated multi-arm polymer having a core and a plurality of polymer arms that have oxyamine end groups and (b) a second multifunctional molecule in the form of a non-polymeric molecule having a non-iodinated core and a plurality of pendant reactive moieties that comprise aldehyde or ketone groups.

As another example, the system may comprise (a) first multifunctional molecule in the form of an iodinated multi-arm polymer having a core and a plurality of polymer arms that have oxyamine end groups and (b) a second multifunctional molecule in the form of a non-polymeric molecule having an iodinated core and a plurality of pendant reactive moieties that comprise aldehyde or ketone groups.

As another example, the system may comprise (a) first multifunctional molecule in the form of an iodinated multi-arm polymer having a core and a plurality of polymer arms that have oxyamine end groups and (b) a second multifunctional molecule in the form of a non-iodinated multi-arm polymer having a core and a plurality of polymer arms that have aldehyde or ketone end groups.

As another example, the system may comprise (a) first multifunctional molecule in the form of an iodinated multi-arm polymer having a core and a plurality of polymer arms that have oxyamine end groups and (b) a second multifunctional molecule in the form of an iodinated multi-arm polymer having a core and a plurality of polymer arms that have aldehyde or ketone end groups.

As another example, the system may comprise (a) a first multifunctional molecule in the form of a non-polymeric molecule having a non-iodinated core and a plurality of pendant reactive moieties that comprise oxyamine groups and (b) a second multifunctional molecule in the form of an iodinated multi-arm polymer having a core and a plurality of polymer arms that have aldehyde or ketone end groups.

As another example, the system may comprise (a) a first multifunctional molecule in the form of a non-polymeric molecule having an iodinated core and a plurality of pendant reactive moieties that comprise oxyamine groups and (b) a second multifunctional molecule in the form of a non-iodinated multi-arm polymer having a core and a plurality of polymer arms that have aldehyde or ketone end groups.

As another example, the system may comprise (a) a first multifunctional molecule in the form of a non-polymeric molecule having an iodinated core and a plurality of pendant reactive moieties that comprise oxyamine groups and (b) a second multifunctional molecule in the form of an iodinated multi-arm polymer having a core and a plurality of polymer arms that have aldehyde or ketone end groups.

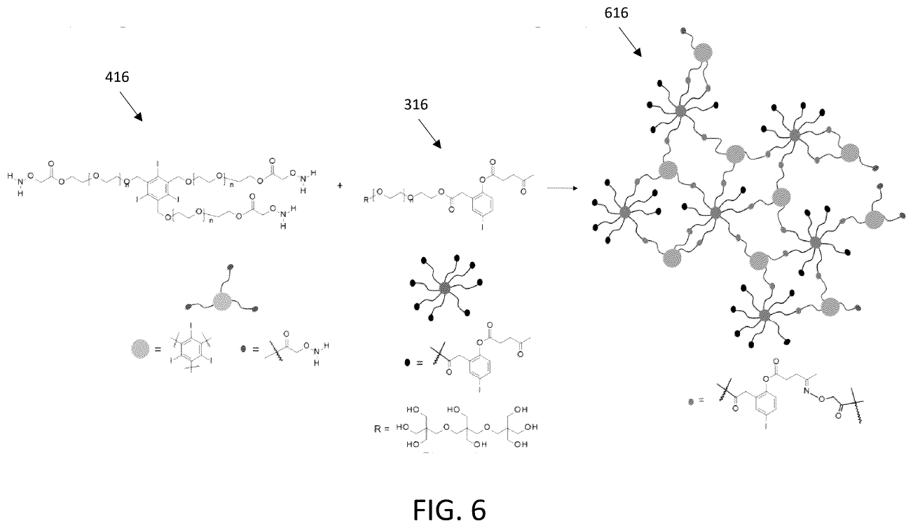

As also noted above, the oxyamine groups of the first multifunctional molecule and the aldehyde or ketone end groups of the second multifunctional molecule in such systems can react via a click reaction to form radiopaque crosslinked reaction products, including radiopaque hydrogels. For example, in one particular embodiment shown in FIG. 6, the iodinated ketone-terminated multi-arm PEG (316) of FIG. 3 is reacted in click reaction with the iodinated oxyamine-terminated multi-arm PEG (416) of FIG. 4 to form a radiopaque crosslinked reaction product (516).

Such crosslinking reactions occur spontaneously in aqueous solutions (e.g., at a pH ranging from 5 to 6.9, without the need for a catalyst while producing water as a reaction by-product and rapidly reaching completion, for example, with crosslinking times ranging from 3 to 10 seconds.

In various embodiments, the radiopaque crosslinked reaction products (including the radiopaque hydrogels) have a radiopacity that is 100 Hounsfield units (HU) or more, beneficially anywhere ranging from 100 HU to 250 HU to 500 HU to 750 HU to 1000 HU to 2500 HU or more.

In some embodiments, the radiopaque crosslinked reaction products break down in-vivo over a period ranging from 2 to 26 weeks.

In some aspects of the present disclosure, systems are provided that are configured to deliver (a) first multifunctional molecule as described hereinabove and (b) a second multifunctional molecule as described hereinabove. The first multifunctional molecule and second multifunctional molecule are comingled such that oxyamine groups of the first multifunctional molecule react and form covalent bonds with the aldehyde or ketone groups of the second multifunctional molecule. Such systems can be used to form crosslinked hydrogels, either in vivo or ex vivo.

In some embodiments, a system is provided that comprises (a) a first composition that comprises a first multifunctional molecule and (b) a second composition that comprises a second multifunctional molecule that forms covalent crosslinks with the first multifunctional molecule. In some embodiments, a third composition in the form of an accelerant composition is also provided.

The first composition may be a first fluid composition comprising the first multifunctional molecule or a first dry composition that comprises the first multifunctional molecule, to which a suitable fluid such as water for injection, saline, etc. can be added to form a first fluid composition. In addition to the first multifunctional molecule, the first composition may further comprise additional agents, including those described below. The first composition may be provided in a suitable reservoir such as a syringe, vial or other reservoir.

The second composition may be a second fluid composition comprising the second multifunctional molecule or a second dry composition that comprises the second multifunctional molecule, to which a suitable fluid such as water for injection, saline, etc. can be added to form a second fluid composition. In addition to the second multifunctional molecule, the second composition may further comprise additional agents, including those described below. The second composition may be provided in a suitable reservoir such as a syringe, vial or other reservoir.

The accelerant composition may be a fluid accelerant composition that is buffered to an acidic pH or a dry composition that comprises a basic buffering composition to which a suitable fluid such as water for injection, saline, etc. can be added to form a fluid accelerant composition that is buffered to an acidic pH. The accelerant composition may further comprise additional agents, including those described below. The accelerant composition may be provided in a suitable reservoir such as a syringe, vial or other reservoir.

In some embodiments, the first multifunctional molecule is initially combined with the second multifunctional molecule at neutral pH at which covalent crosslinking between the first and second multifunctional molecules are suppressed. Then, when covalent crosslinking is desired, a pH of the mixture of the first multifunctional molecule and the second multifunctional molecule is changed from a neutral pH to an acidic pH, leading to accelerated covalent crosslinking between the same.

In some embodiments, a system is provided wherein a precursor fluid composition is prepared, which comprises a mixture of the first multifunctional molecule and the second multifunctional molecule and is buffered to neutral pH. The precursor fluid composition may further comprise additional agents, including those described below. The system also includes a fluid accelerant composition that comprises an acidic buffer. The fluid accelerant composition may further comprise additional agents, including those described below. The first composition (precursor fluid composition) and the second composition (fluid accelerant composition) may then be combined to change a pH of the mixture of the first multifunctional molecule and the second multifunctional molecule from a neutral pH to an acidic pH, thereby forming covalently crosslinked hydrogels, either in vivo or ex vivo.

Additional agents for use in the present disclosure include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imagable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second fluid compositions to a subject. Preferred subjects include mammalian subjects, particularly human subjects.

In some embodiments, the system may include a delivery device that comprises a first reservoir that contains a first fluid composition that comprises a first multifunctional molecule as described above and a second reservoir that contains a second fluid composition that comprises a second multifunctional molecule as described above.

In some embodiments, the system may include a delivery device that comprises (a) a first reservoir that contains a first fluid composition in the form of a precursor fluid composition that comprises a first multifunctional molecule and a second multifunctional molecule as described above (b) a second reservoir that contains a second fluid composition in the form of a fluid accelerant composition as described above.

In either case, when the first and second fluid compositions are mixed, crosslinking occurs or is accelerated between the first multifunctional molecule and the second multifunctional molecule. During operation, the first fluid composition and second fluid composition are dispensed from the first and second reservoirs and combined, whereupon the first and second multifunctional molecules crosslink with one another to form a crosslinked hydrogel.

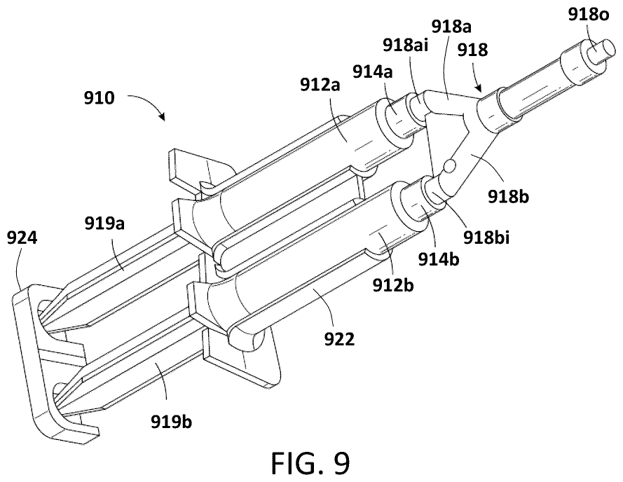

In particular embodiments, and with reference to FIG. 9 the system may include a delivery device 910 that comprises a double-barrel syringe, which includes a first barrel 912a having a first barrel outlet 914a, which first barrel contains the first fluid composition, a first plunger 919a that is movable in the first barrel 912a, a second barrel 912b having a second barrel outlet 914b, which second barrel 912b contains the second fluid composition, and a second plunger 919b that is movable in the second barrel 912b. In some embodiments, the device 910 may further comprise a mixing section 918 having a first mixing section inlet 918ai in fluid communication with the first barrel outlet 914a, a second mixing section inlet 918bi in fluid communication with the second barrel outlet, and a mixing section outlet 918o. Also shown are a syringe holder 922 configured to hold the first and second syringe barrels 912a, 912b, in a fixed relationship and a plunger cap 924 configured to hold the first and second plungers 919a, 919b in a fixed relationship.

In some embodiments, the delivery device may further comprise a cannula or catheter tube that is configured to receive the first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture initially may be in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique such as ultrasound or an X-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked product of the first and second compositions, a procedure to implant a tissue regeneration scaffold comprising a crosslinked product of the first and second compositions, a procedure to implant a tissue support comprising a crosslinked product of the first and second compositions, a procedure to implant a tissue bulking agent comprising a crosslinked product of the first and second compositions, a procedure to implant an embolic composition comprising a crosslinked product of the first and second compositions, a procedure to implant a lifting agent comprising a crosslinked product of the first and second compositions, a procedure to introduce a left atrial appendage closure composition comprising a crosslinked product of the first and second compositions, a procedure to implant a therapeutic-agent-containing depot comprising a crosslinked product of the first and second compositions, a tissue augmentation procedure comprising implanting a crosslinked product of the first and second compositions, a procedure to introduce a crosslinked product of the first and second compositions between a first tissue and a second tissue to space the first tissue from the second tissue.

The first and second fluid compositions, fluid admixtures of the first and second fluid compositions, or the crosslinked products of the first and second compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked hydrogel particles formed using the above and other techniques may varying widely in size, for example, having an average size ranging from 50 to 950 microns.

In addition to a crosslinked reaction product as described herein, crosslinked hydrogel compositions in accordance with the present disclosure may further contain additional agents, including therapeutic agents, imaging agents, colorants, tonicity adjusting agents, suspension agents, wetting agents, and pH adjusting agents as described above.

Crosslinked hydrogel compositions in accordance with the present disclosure include injectable fluid suspensions of crosslinked hydrogel particles.

In various embodiments, kits are provided that include one or more delivery devices for delivering the crosslinked hydrogel to a subject. Such systems may include one or more of the following: a syringe barrel, which may or may not contain a crosslinked hydrogel as described herein; a vial, which may or may not contain a crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel may be provided in dry form (e.g., powder form) or in a form that is ready for injection, such as an injectable hydrogel form (e.g., a suspension of crosslinked hydrogel particles).

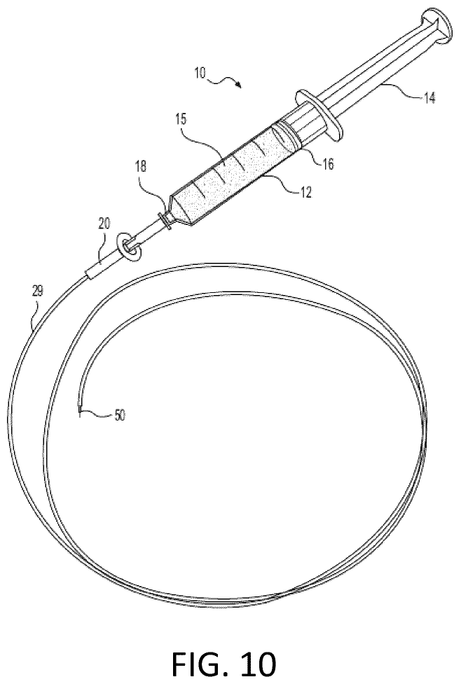

FIG. 10 illustrates a syringe 10 providing a reservoir for a crosslinked hydrogel composition as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

The crosslinked hydrogel compositions described herein can be used for a number of purposes.

For example, crosslinked hydrogel compositions can be injected to provide spacing between tissues, crosslinked hydrogel compositions can be injected (e.g., in the form of blebs) to provide fiducial markers, crosslinked hydrogel compositions can be injected for tissue augmentation or regeneration, crosslinked hydrogel compositions can be injected as a filler or replacement for soft tissue, crosslinked hydrogel compositions can be injected to provide mechanical support for compromised tissue, crosslinked hydrogel compositions be injected as a scaffold, and/or crosslinked hydrogel compositions can be injected as a carrier of therapeutic agents in the treatment of diseases and cancers and the repair and regeneration of tissue, among other uses.

The crosslinked hydrogel compositions of the present disclosure may be used in a variety of medical procedures, including the following, among others: a procedure to implant a fiducial marker comprising a crosslinked hydrogel, a procedure to implant a tissue regeneration scaffold comprising a crosslinked hydrogel, a procedure to implant a tissue support comprising a crosslinked hydrogel, a procedure to implant a tissue bulking agent comprising a crosslinked hydrogel, a procedure to implant a therapeutic-agent-containing depot comprising a crosslinked hydrogel, a tissue augmentation procedure comprising implanting a crosslinked hydrogel, a procedure to introduce a crosslinked hydrogel between a first tissue and a second tissue to space the first tissue from the second tissue.

The crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

After administration, the crosslinked hydrogel compositions of the present disclosure can be imaged using a suitable imaging technique such as ultrasound or an X-ray-based imaging technique, such as computerized tomography or X-ray fluoroscopy.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

It should be understood that this disclosure is, in many respects, only illustrative and that changes may be made in details without exceeding the scope of the disclosure. This may include, to the extent that it is appropriate, the use of any of the features of one embodiment being used in other embodiments.

1. A system for forming a crosslinked reaction product comprising (a) a first composition that comprises a first multifunctional molecule that comprises a plurality of first reactive moieties that comprise oxyamine groups and (b) a second composition that comprises a second multifunctional molecule that comprises a plurality of second reactive moieties that comprise aldehyde or ketone groups, wherein at least one of the first and second multifunctional molecules is a multi-arm polymer and wherein at least one of the first and second multifunctional molecules comprises one or more covalently attached iodine groups.

2. The system of claim 1, wherein the first reactive moieties further comprise hydrolysable linkages through which the oxyamine groups are linked to a remainder of the first multifunctional molecule and/or wherein the second reactive moieties further comprise hydrolysable linkages through which the aldehyde or ketone groups are linked to a remainder of the first multifunctional molecule.

3. The system of claim 1, wherein at least one of the first and second multifunctional molecules comprises covalently attached iodine groups in an iodinated core.

4. The system of claim 1, wherein at least one of the first and second multifunctional molecules comprises covalently attached iodine groups in a residue of an iodo-substituted cyclic ester compound.

5. The system of claim 1, (a) wherein the first multifunctional molecule is an iodinated multi-arm polymer having a core and a plurality of polymer arms that have oxyamine end groups and (b) wherein the second multifunctional molecule is a non-iodinated multi-arm polymer having a core and a plurality of polymer arms that have aldehyde or ketone end groups, an iodinated multi-arm polymer having a core and a plurality of polymer arms that have aldehyde or ketone end groups, a non-polymeric molecule having a non-iodinated core and a plurality of pendant reactive moieties that comprise aldehyde or ketone groups, or a non-polymeric molecule having an iodinated core and a plurality of pendant reactive moieties that comprise aldehyde or ketone groups.

6. The system of claim 1, (a) wherein the first multifunctional molecule is a non-iodinated multi-arm polymer having a core and a plurality of polymer arms that have oxyamine end groups and (b) wherein the second multifunctional molecule is an iodinated multi-arm polymer having a core and a plurality of polymer arms that have aldehyde or ketone end groups or a non-polymeric molecule having an iodinated core and a plurality of pendant reactive moieties that comprise aldehyde or ketone groups.

7. The system of claim 1, (a) wherein the first multifunctional molecule is a non-polymeric molecule having an iodinated core and a plurality of pendant reactive moieties that comprise oxyamine groups and (b) wherein the second multifunctional molecule is a non-iodinated multi-arm polymer having a core and a plurality of polymer arms that have aldehyde or ketone end groups or an iodinated multi-arm polymer having a core and a plurality of polymer arms that have aldehyde or ketone end groups.

8. The system of claim 1, (a) wherein the first multifunctional molecule is a non-polymeric molecule having a non-iodinated core and a plurality of pendant reactive moieties that comprise oxyamine groups and (b) wherein the second multifunctional molecule is an iodinated multi-arm polymer having a core and a plurality of polymer arms that have aldehyde or ketone end groups.

9. The system of claim 4, wherein the iodinated core comprises one or more iodinated aromatic groups.

10. The system of claim 4, wherein the iodinated core comprises an iodinated polyol residue.

11. The system of claim 1, comprising the first composition in a first container and the second composition in a second container.

12. The system of claim 11, wherein the first and second containers are independently selected from vials and syringes.

13. The system of claim 11, further comprising a delivery device that is configured to deliver the first and second compositions.

14. The system of claim 13, wherein the delivery device comprises a dual barrel syringe.

15. A crosslinked network formed by combining (a) a first multifunctional molecule that comprises a plurality of first reactive moieties that comprise oxyamine groups and (b) a second multifunctional molecule that comprises a plurality of second reactive moieties that comprise aldehyde or ketone groups, wherein at least one of the first and second multifunctional molecules is a multi-arm polymer and wherein at least one of the first and second multifunctional molecules comprises one or more covalently attached iodine groups.

16. The crosslinked network of claim 15, wherein the crosslinked network is a hydrogel.

17. The crosslinked network of claim 15, wherein the crosslinked network has a radiopacity that is greater than 250 Hounsfield units.

18. The crosslinked network of claim 15 in the form of a suspension of particles of the crosslinked network.

19. The crosslinked network of claim 18, wherein the suspension of particles is provided in a syringe barrel.

20. A method of treatment comprising administering to a subject a mixture that comprises (a) a first multifunctional molecule that comprises a plurality of first reactive moieties that comprise oxyamine groups and (b) a second multifunctional molecule that comprises a plurality of second reactive moieties that comprise aldehyde or ketone groups, wherein at least one of the first and second multifunctional molecules is a multi-arm polymer and wherein at least one of the first and second multifunctional molecules comprises one or more covalently attached iodine groups.
